(19) United States
(12) Plant Patent Application Publication
(10) Pub. No.: US 2002/0120966 P1
(43) Pub. Date: Aug. 29, 2002

(54) PEACH TREE NAMED 'SUPECHTHIRTEEN'

(76) Inventors: Carlos D. Fear, Aptos, CA (US); Bruce D. Mowrey, La Selva Beach, CA (US); David W. Cain, Bakersfield, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/792,970

(22) Filed: Feb. 26, 2001

(51) Int. Cl. A01H 5/00

(52) U.S. Cl. PLT/197

(57) ABSTRACT

A new and distinct peach variety that possesses early-ripening fruit with delicate flavor, hardiness and a low winter chilling requirement.

### BACKGROUND AND SUMMARY OF THE INVENTION

This invention relates to the discovery and asexual propagation of a new variety of peach, Prunus persica cv. ‘Supechthirteen’. The new variety was first hybridized by Carlos D. Fear in May 13, 1991. The new variety was selected and evaluated by Bruce D. Mowrey and David W. Cain, near Wasco, Kern County, Calif., the variety being originated by hybridization. The new variety is characterized by its early-ripening fruit with delicate flavor, its hardiness and its low winter chilling requirement.

The seed parent is ‘Flordaprince’ (unpatented) and the pollen parent is ‘Earligrande’ (unpatented). The parent varieties were first crossed in February 1989. The date of sowing was November 1989, with the date of first flowering being February 1991. The new peach variety was first asexually propagated by Bruce D. Mowrey in June 1991, near Wasco, Kern County, Calif. by budding onto Nemared rootstock.

The new peach variety ‘Supechthirteen’ may be distinguished from presently available low-chilling cultivars in commerce by the following combination of characteristics: the new peach variety cv. ‘Supechthirteen’ most nearly resembles ‘Flordaprince’. It differs from ‘Flordaprince’ by ripening approximately 7 days before ‘Flordaprince’ and has more (80-100%) red overcolor compared to ‘Flordaprince’ (70-90%) and the red overcolor is more evenly distributed and less striped than with ‘Flordaprince’. The new cultivar vaguely resembles ‘Tropic Beauty’ but ripens approximately 17 days before ‘Tropic Beauty’.

The new peach cultivar, ‘Supechthirteen’ may be distinguished from its pollen parent ‘Earligrande’ (unpatented) in the following characteristics: the new cultivar ripens approximately 10 days before ‘Earligrande’. The new cultivar has a lower winter chilling requirement, requiring approximately 150 hours of temperature below 45° .F vs. 250 hours for ‘Earligrande’. The new cultivar also has more (80-100%) external red blush than ‘Earligrande’ (40-60%).

The new peach variety cv. ‘Supechthirteen’ has been shown to maintain its distinguishing characteristics through successive asexual propagations, for example, by budding. The first propagation was performed near Wasco, Kern County, Calif. on June 1991 by Bruce D. Mowrey by budding onto Nemared rootstock.

### BRIEF DESCRIPTION OF THE FIGURE

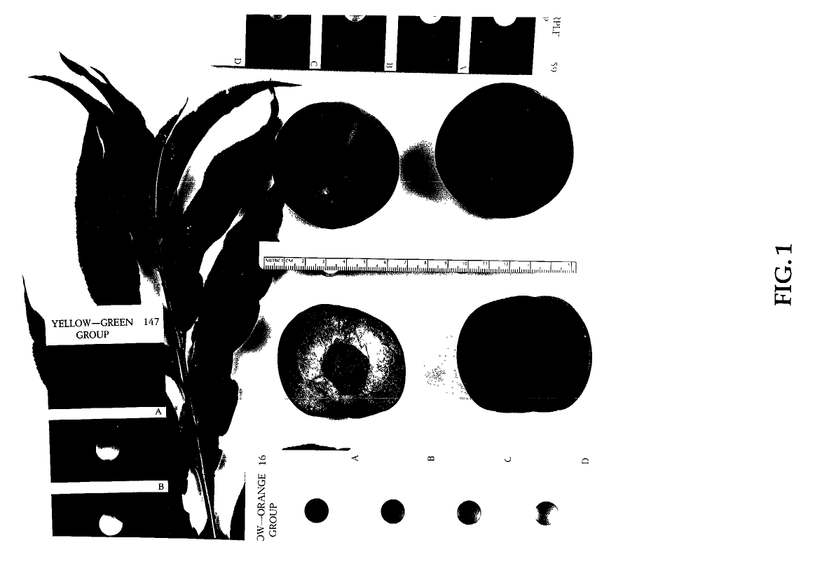

The accompanying drawing in FIG. 1 illustrates in full color.

### DETAILED BOTANICAL DESCRIPTION OF THE INVENTION

Throughout this specification, color names beginning with a small letter signify that the name of that color, as used in common speech, is aptly descriptive. Color names beginning with a capital letter designate values based upon the R.H.S. Colour Chart, published by The Royal Horticultural Society, London, England.

The descriptive matter which follows pertains to ‘Supechthirteen’ plants grown in the vicinity of Wasco, Kern County, Calif. with bloom and ripe dates also given for low chill sites near Mecca, Riverside County, Calif., during 2000, and is believed to apply to plants of the variety grown under similar conditions of soil and climate elsewhere:

### TREE

General: Trees were three years old and were maintained at a height of about ten feet and a canopy diameter of about ten feet by annual prunings.

Size.—medium.

Vigor.—medium.

Habit.—semi-upright.

Density of foliage.—medium.

Shape.—vase formed.

Hardiness.—hardy near Wasco, Calif.

Productivity.—medium productive.

Fruit bearing.—regular bearer.

Root stock.—Nemared.

Trunk: Trunk is round in outline and of medium circumference, averaging 60 cm at 30 cm above ground level on 3-year-old trees.

Shape.—medium.

Surface texture.—smooth—medium.

Color.—about Grey-Brown group 199B.

Branches: Branches are round in outline and average 20 cm in circumference on 3-year-old trees as measured 30 cm distal from the branch juncture with the trunk.

Shape.—medium—slender.

Surface texture.—smooth—medium.

Surface appearance.—semi-glossy—dull.

Lenticels:

Number.—medium—few.

Size.—large—medium.

Density.—1.6/cm².

### LEAVES

General:

Average length.—15.3 cm.

Average Width.—3.4 cm.

Size.—medium.

Outline.—lanceolate.

Profile.—up folded.

Leaf blade tip.—curved downwardly.

Angle of tip.—acute.

Margin.—finely serrate.

Undulation of margin.—slight.

Apex.—acuminate.

Base.—V-shaped.

Thickness.—medium.

Upper Surface:

Color.—about Yellow-Green group 147A.

Glossiness.—medium.

Pubescense.—absent.

Surface texture.—rugose.

Surface appearance.—semi-glossy.

Lower Surface:

Color.—about Yellow-Green group 147B.

Glossiness.—weak.

Pubescense.—absent.

Surface texture.—rugose.

Surface appearance.—dull.

Petiole:

Length.—medium—short, about 1.1 cm.

Thickness.—medium, about 2 mm.

Glands:

Average number.—4.3.

Positioning.—on both leaf base and petiole.

Size.—medium.

Shape.—reniform.

Color.—about Yellow-Green group 153D.

Stipules:

Persistence.—fall off.

Wood (Leaf) Buds:

Shape.—ovoid.

Size.—small—medium.

Position relative to shoot.—adpressed.

Support.—decurrent.

Size.—small.

Time of bud burst.—medium in comparison to other low chill (150-200 chill hour) varieties.

### FLOWERING SHOOTS:

Anthocyanin coloration.—present.

Intensity.—medium.

Thickness.—thin—medium, about 0.4 cm.

Internode length.—medium, about 1.7 cm, midway on flowering shoots.

Density of buds.—medium.

Distribution.—on one-year-old shoots.

Ratio of wood (leaf) buds to flowering shoots.—1:2.

### FLOWERS

Flower-Buds:

Hardiness.—hardy in central California.

Size.—medium.

Length.—medium.

Shape.—plump.

Positioning.—free.

Pubescence.—pubescent.

Color.—about Greyed-Orange group 165A.

General:

Date of first bloom.—Jan. 25, 2000 at low chill site in Riverside County, near Mecca, Calif. Feb. 3, 2000 at medium high chill site in Wasco, Kern County, Calif.

Date of full bloom.—Jan. 30, 2000 at low chill site in Riverside County, near Mecca, Calif.; Feb. 8, 2000 at medium high chill site in Wasco, Kern County, Calif.

Time of bloom.—medium, as compared with similar low chill varieties in the growing area of Wasco, Kern County and Mecca, Riverside County, Calif.

Duration of bloom.—medium, about 10 days.

Size (diameter of the fully open flower).—small, about 2.6 cm.

Shape.—campanulate.

Petals.—free.

Color (fully opened flower).—about 62B.

Peduncle:

Length.—short.

Thickness.—medium.

Pubescence.—absent.

Receptacle:

Depth.—medium.

Pubescence of inner surface (at pink bud stage).—pubescent.

Pubescence of outer surface.—absent.

Sepals:

Positioning.—adpressed to petals.

Shape.—elliptic.

Pubescence of inner surface.—absent.

Pubescence of outer surface.—pubescent.

Frequency of flowers with double sepals.—none.

Petals:

Frequency of flowers with double petals.—none.

Size.—small, about 0.7 cm. width.

Shape.—elliptical.

Claw length.—short.

Margin waviness.—absent.

Base angle.—medium.

Division of upper margin.—entire.

Pubescence of inner surface.—absent.

Pubescence of outer surface.—absent.

Color (inner surface).—about 62B.

Color (outer surface).—about 62B.

Stigma:

Position (as compared with anthers).—level.

Anthers:

Color (just before dehiscence).—about Red 39A on outer edges and about Yellow 14B in recesses.

Pollen:

Color.—about 14C.

Stamens:

Position.—perigynous.

Pistil:

Frequency of supplementary pistils.—few.

Number.—sometimes more than one.

Ovary:

Pubescence.—pubescent.

Density of hairs.—dense.

Style:

Pubescence (of base).—medium.

Density of hairs.—medium.

### FRUIT

General:

Maturity when described.—firm, ripe.

Date.—Apr. 8, 2000 at the low-chill site near Mecca, Riverside County, Calif.; May 2, 2000 at the medium-high chill site near Wasco, Kern County, Calif.

Size.—medium, about 6 cm, about 120 g.

Axial diameter.—about 6 cm.

Transverse in suture plane.—6.5 cm.

At right angles to suture plane.—6 cm.

Form.—uniform, rounded, and slightly flattened toward tip. Tip tends to be slightly recessed.

Position of maximum diameter.—towards middle.

Symmetry about the suture.—symmetric.

Season ripening.—early.

Use.—fresh market.

Keeping quality.—medium.

Resistance to.—Insects: good. Diseases: good. Shipping quality: medium.

Suture: an inconspicuous line extending from base to apex along one side.

Ventral Surface:

Shape.—rounded slightly.

Lips.—equal.

Depression of apex.—indistinct.

Pistil base.—not persisting.

Pubescence at apex.—medium.

Density of apex pubescence.—medium.

Stem Cavity:

Shape.—flaring, circular; with suture showing on one side.

Depth.—1.3 cm.

Breadth.—2.8 cm.

Base:

Shape.—rounded.

Apex:

Shape.—slightly depressed.

Pistil Point:

Shape.—slightly depressed.

Stem:

Length.—0.8 cm, stout, glabrous.

Adherence to stone.—strong.

Skin:

Thickness.—medium.

Texture.—medium.

Reticulation.—absent.

Roughness.—absent.

Tenacity.—tenacious to flesh.

Tendency to crack.—none in wet season.

Color.—Ground color: 8C; Overcolor: 59A.

Down (pubescence).—moderate to scant, short.

Flesh:

Color.—Near skin: about Red-Purple 59B; Near pit: about Yellow 8B.

Amygdalin.—wanting.

Juice.—moderate.

Sugar content.—low to medium.

Texture.—medium, coarse, melting.

Fibers.—abundant, coarse.

Ripens.—earliest at apex.

Flavor.—delicate.

Eating quality.—fair—good for early season fruit.

Stone/flesh ratio.—1 cm diameter/3.2 cm diameter.

### STONE

General:

Adherence to flesh.—cling, adheres to flesh over entire surface.

Fibers.—long.

Size.—medium—small.

Length.—2.4 cm.

Width along suture plane.—2.3 cm.

Width at right angle to suture plane.—1.8 cm.

Width of stalk end.—0.4 cm.

Angle of stalk end.—obtuse.

Form (profile).—oval.

Form (ventral view).—globular.

Base.—oblique.

Hilum.—oval.

Apex.—pointed.

Position of maximum breadth.—middle.

Sides.—equal.

Surface.—regularly furrowed near base; pitted throughout.

Outgrowing keel.—partly developed.

Ridges.—rounded towards base.

Pits.—elongated.

Ventrical edge.—thin with slight wing toward base.

Dorsal edge.—narrow.

Color of stone.—about Greyed-Orange 165D.

Tendency to split.—slight in wet season.

What is claimed is:

1. A new and distinct variety of peach cv. ‘Supechthirteen’ as herein illustrated and described.
